(19) United States
(12) Patent Application Publication
(10) Pub. No.: US 2025/0352157 A1
(43) Pub. Date: Nov. 20, 2025

(54) X-RAY CT APPARATUS AND INJECTION APPARATUS

(71) Applicant: CANON MEDICAL SYSTEMS CORPORATION, Otawara-shi (JP)

(72) Inventor: Masaharu TSUYUKI, Nasushiobara (JP)

(73) Assignee: CANON MEDICAL SYSTEMS CORPORATION, Otawara-shi (JP)

(21) Appl. No.: 19/208,872

(22) Filed: May 15, 2025

(30) Foreign Application Priority Data
May 15, 2024 (JP) 2024-079595

(51) Int. Cl. A61B 6/00; A61B 6/03

(52) U.S. Cl. CPC A61B 6/481; A61B 6/032; A61B 6/4447

(57) ABSTRACT

An X-ray CT apparatus according to an embodiment includes a gantry, a supporter, and an injection path. The gantry includes a hollow into which a subject is inserted, an X-ray tube that emits X-rays to the hollow, and an X-ray detector that detects X-rays passed through the hollow. The supporter supports the gantry, and moves the gantry in a vertical direction. Through the injection path, a medical fluid is injected into the subject from at least one of an upper side and a lower side in the vertical direction of the gantry.

### CROSS-REFERENCE TO RELATED APPLICATIONS

This application is based upon and claims the benefit of priority from Japanese Patent Application No. 2024-079595, filed on May 15, 2024; the entire contents of all of which are incorporated herein by reference.

### FIELD

Embodiments described herein relate generally to an X-ray CT apparatus and an injection apparatus.

### BACKGROUND

Conventionally, an X-ray Computed Tomography (CT) apparatus performs photographing by injecting a contrast medium into a subject to add contrast to an image or emphasize specific tissues. The contrast medium is injected into the subject by an injection apparatus via a tube. At this point, if the tube through which the contrast medium passes is present in a photographing region, the X-ray CT apparatus may produce artifacts due to the contrast medium.

Thus, a health professional places a subject in a lying position on a table of the X-ray CT apparatus, and injects the contrast medium at the vicinity of an elbow of the subject raising his/her arm. The health professional then photographs the subject in the foot-first position, that is, the feet of the subject are put into the X-ray CT apparatus first. Due to this, the X-ray CT apparatus can photograph the subject in a state in which the tube through which the contrast medium passes is not disposed in the photographing region. In other words, the X-ray CT apparatus can prevent artifacts caused by the contrast medium passing through the tube.

In recent years, X-ray CT apparatuses for photographing a subject in a standing position or a sitting position have appeared. For such X-ray CT apparatuses, there is a demand for performing photographing by injecting the contrast medium into the subject.

However, the subject is in a standing position or a sitting position. Thus, when a conventional method is used, the tube through which the contrast medium passes is disposed inside the photographing region.

### BRIEF DESCRIPTION OF THE DRAWINGS

FIG. 1 is a diagram illustrating an example of a configuration of an X-ray CT apparatus according to a first embodiment;

FIG. 2 is a diagram illustrating an example of an injection route of a medical fluid for a subject in the X-ray CT apparatus according to the first embodiment;

FIG. 3 is a diagram illustrating an example of a gantry according to a first modification of the first embodiment;

FIGS. 4A to 4C are diagrams illustrating an example of a gantry according to a second modification of the first embodiment;

FIGS. 5A to 5C are diagrams illustrating an example of the gantry according to the second modification of the first embodiment;

FIGS. 6A to 6C are diagrams illustrating an example of the gantry according to the second modification of the first embodiment;

FIGS. 7A to 7C are diagrams illustrating an example of the gantry according to the second modification of the first embodiment;

FIG. 8 is a diagram illustrating an example of a configuration of an X-ray CT apparatus according to a second embodiment;

FIG. 9 is a diagram illustrating an example of a configuration of an injection apparatus;

FIG. 10 is a diagram illustrating an example of a connection configuration of tubes of the injection apparatus according to the second embodiment;

FIG. 11 is a diagram illustrating an example of a connection configuration of tubes of an injection apparatus according to a first modification of the second embodiment;

FIG. 12 is a diagram illustrating an example of a connection configuration of tubes of an injection apparatus according to a second modification of the second embodiment;

FIG. 13 is a diagram illustrating an example of a connection configuration of tubes of an injection apparatus according to a third modification of the second embodiment;

FIG. 14 is a diagram illustrating an example of attachment of a replacement assisting member according to a fourth modification of the second embodiment; and

FIG. 15 is a diagram illustrating an example of external appearance of the replacement assisting member.

### DETAILED DESCRIPTION

An X-ray CT apparatus according to an embodiment includes a gantry, a supporter, and an injection path. The gantry includes a hollow into which a subject is inserted, an X-ray tube that emits X-rays to the hollow, and an X-ray detector that detects X-rays passed through the hollow. The supporter supports the gantry, and moves the gantry in a vertical direction. Through the injection path, a medical fluid is injected into the subject from at least one of an upper side and a lower side in the vertical direction of the gantry.

The following describes the X-ray CT apparatus and an injection apparatus according to the present embodiment with reference to the drawings. In the following embodiment, redundant description will be appropriately omitted assuming that portions denoted by the same reference numeral perform the same operation.

### First Embodiment

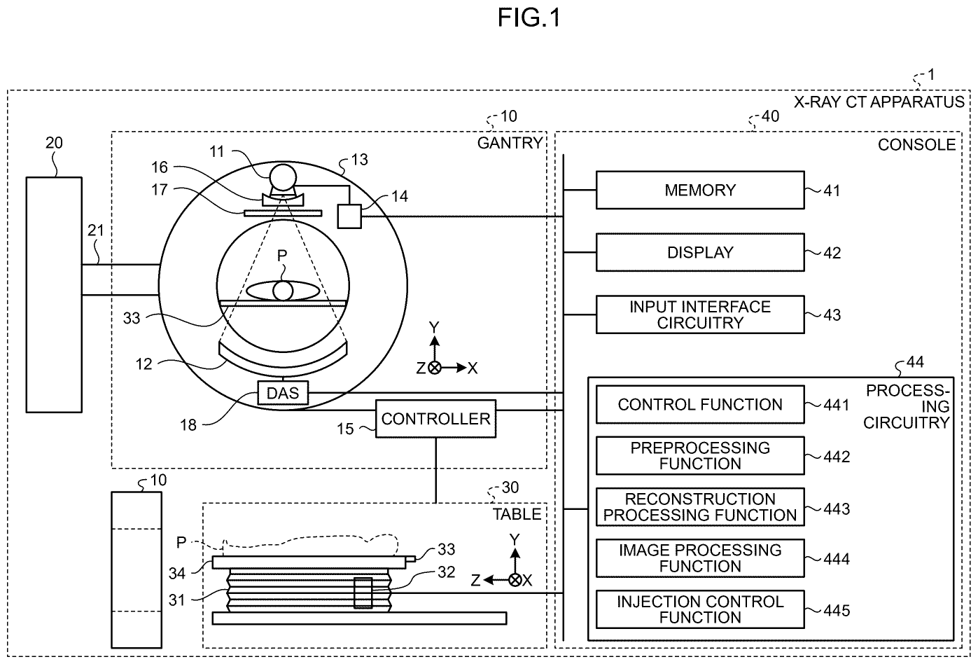

FIG. 1 is a diagram illustrating an example of a configuration of an X-ray CT apparatus 1 according to a first embodiment. As illustrated in FIG. 1, the X-ray CT apparatus 1 includes a gantry 10, a stand 20, a table 30, and a console 40. That is, the X-ray CT apparatus 1 can photograph not only a subject P in a lying position but also the subject P in a standing position and a sitting position. Alternatively, the X-ray CT apparatus 1 may photograph the subject P in a standing position or a sitting position.

Herein, in FIG. 1, a rotation axis of a rotary frame 13 in a non-tilted state or a longitudinal direction of a table-top 33 of the table 30 is assumed to be a Z-axis direction. An axis direction that is orthogonal to the Z-axis direction and horizontal to a floor surface is assumed to be an X-axis direction. An axis direction that is orthogonal to the Z-axis direction and the X-axis direction and perpendicular to the floor surface is assumed to be a Y-axis direction. FIG. 1 depicts the gantry 10 from a plurality of directions for explanation, and illustrates a case in which the X-ray CT apparatus 1 includes the one gantry 10.

The gantry 10 includes an X-ray tube 11, an X-ray detector 12, the rotary frame 13, an X-ray high-voltage apparatus 14, a control apparatus 15, a wedge 16, a collimator 17, and a data acquisition system (DAS) 18. The gantry 10 is also referred to as a gantry. The gantry 10 has an opening through which the subject P is inserted. That is, the gantry 10 includes a hollow into which the subject P is inserted, the X-ray tube 11 that emits X-rays to the hollow, and the X-ray detector 12 that detects X-rays that are emitted by the X-ray tube 11 and passed through the hollow. The gantry 10 is supported by the stand 20. The gantry 10 is covered by a housing. An inner part of the gantry 10 is an inner space of the housing.

The X-ray tube 11 is a vacuum tube including a cathode (filament) that generates thermoelectrons, and an anode (target) that generates X-rays when the thermoelectrons collide therewith. When high voltage is applied from the X-ray high-voltage apparatus 14, the X-ray tube 11 emits thermoelectrons from the cathode toward the anode to generate X-rays to be emitted to the subject P. Examples of the X-ray tube 11 include a rotating anode-type X-ray tube that generates X-rays by emitting thermoelectrons to a rotating anode.

The X-ray detector 12 detects X-rays that have been emitted from the X-ray tube 11 and passed through the subject P, and outputs a signal corresponding to a dose of the detected X-rays to the DAS 18. The X-ray detector 12 includes, for example, a plurality of detection element arrays in which a plurality of detection elements are arranged in a channel direction along one arc centered on a focal point of the X-ray tube 11. The X-ray detector 12 has, for example, a structure in which the detection element arrays are arranged in a column direction (a slice direction, a row direction), the detection element arrays in which the detection elements are arranged in the channel direction.

For example, the X-ray detector 12 is a detector of an indirect conversion type including a grid, a scintillator array, and an optical sensor array. The scintillator array includes a plurality of scintillators. The scintillator includes a scintillator crystal that outputs light with a photon quantity corresponding to an incident X-ray dose. The grid includes an X-ray shielding plate that is disposed on a surface on an X-ray incident side of the scintillator array and absorbs scattered X-rays. The grid may also be referred to as a collimator (a one-dimensional collimator or a two-dimensional collimator). The optical sensor array has a function of converting light into an electric signal corresponding to an amount of light from the scintillator, and includes, for example, an optical sensor such as a photodiode. Alternatively, the X-ray detector 12 may be a detector of a direct conversion type including a semiconductor element that converts incident X-rays into an electric signal.

The rotary frame 13 is a frame having an annular shape that supports the X-ray tube 11 and the X-ray detector 12 to be opposed to each other, and rotates the X-ray tube 11 and the X-ray detector 12 by the control apparatus 15. For example, the rotary frame 13 is a casting made of aluminum as a material. The rotary frame 13 can further support the X-ray high-voltage apparatus 14, the wedge 16, the collimator 17, the DAS 18, and the like in addition to the X-ray tube 11 and the X-ray detector 12. Additionally, the rotary frame 13 can further support various configurations not illustrated in FIG. 1. The various configurations supported by the rotary frame 13 will be described later. The rotary frame 13 is also referred to as a rotating base, a rotating body, or the like. In the gantry 10, the rotary frame 13 and portions that rotate and move together with the rotary frame 13 are also referred to as rotation units.

The X-ray high-voltage apparatus 14 includes electric circuitry such as a transformer and a rectifier, and includes a high-voltage generation apparatus that generates high voltage to be applied to the X-ray tube 11, and an X-ray control apparatus that controls output voltage corresponding to X-rays generated by the X-ray tube 11. The high-voltage generation apparatus may employ a transformer system, or an inverter system. The X-ray high-voltage apparatus 14 may be disposed on the rotary frame 13, or may be disposed on a fixed frame (not illustrated).

The control apparatus 15 includes processing circuitry including a central processing unit (CPU) and the like, and a driving mechanism such as a motor and an actuator. The control apparatus 15 receives an input signal from an input interface circuitry 43 (described later), and performs operation control for the gantry 10 and the table 30. For example, the control apparatus 15 performs control for rotation of the rotary frame 13, tilt of the gantry 10, and operations of the table 30 and the table-top 33. By way of example, as the control for tilting the gantry 10, the control apparatus 15 rotates the rotary frame 13 about an axis parallel with the X-axis direction based on inclination angle (tilt angle) information that has been input. The control apparatus 15 may be disposed on the gantry 10, or may be disposed on the console 40.

The wedge 16 is a filter for adjusting a dose of X-rays emitted from the X-ray tube 11. Specifically, the wedge 16 is a filter that transmits and attenuates X-rays emitted from the X-ray tube 11 so that distribution of the X-rays emitted from the X-ray tube 11 to the subject P becomes predetermined distribution. For example, the wedge 16 is a wedge filter or a bow-tie filter, which is a filter obtained by processing aluminum and the like to have a predetermined target angle and a predetermined thickness.

The collimator 17 is a lead plate and the like for narrowing an irradiation range of X-rays transmitted through the wedge 16, and forms a slit by combining a plurality of lead plates and the like. The collimator 17 may also be referred to as an X-ray aperture. FIG. 1 illustrates a case in which the wedge 16 is disposed between the X-ray tube 11 and the collimator 17, but the collimator 17 may be disposed between the X-ray tube 11 and the wedge 16. In this case, the wedge 16 transmits and attenuates X-rays emitted from the X-ray tube 11 having an irradiation range limited by the collimator 17.

The DAS 18 collects signals of X-rays detected by the detection elements included in the X-ray detector 12. For example, the DAS 18 includes an amplifier that performs amplification processing on an electric signal output from each detection element, and an A/D converter that converts the electric signal into a digital signal, and generates detection data.

The detection data generated by the DAS 18 is transmitted, by optical communication, from a transmitter including a light emitting diode (LED) disposed on the rotary frame 13 to a receiver including a photodiode disposed at a non-rotary portion (for example, a fixed frame and the like, not illustrated in FIG. 1) of the gantry 10, and transferred to the console 40. Herein, the non-rotary portion is a fixed frame and the like that support the rotary frame 13 in a rotatable manner, for example. A method for transmitting data from the rotary frame 13 to the non-rotary portion of the gantry 10 is not limited to the optical communication. Any noncontact-type data transmission scheme may be employed, or a contact-type data transmission scheme may be employed.

The stand 20 supports the gantry 10, and moves the gantry 10 in a vertical direction. The stand 20 is an example of a supporter. More specifically, the stand 20 is fixed to a floor surface of a room in which the gantry 10 is installed. The stand 20 also includes a coupling unit 21 that is coupled with the gantry 10. The coupling unit 21 is an example of a coupler. The coupling unit 21 supports the gantry 10 to be rotatable in a tilt direction. The stand 20 includes a first driving unit that moves the coupling unit 21 in the vertical direction, and a second driving unit that rotates the coupling unit 21 about a direction substantially parallel with the X-axis direction as a rotation axis. The stand 20 drives the first driving unit to move the coupling unit 21 in the vertical direction. The stand 20 also drives the second driving unit to rotate the coupling unit 21 about a direction substantially parallel with the floor surface as a rotation axis. Due to this, the coupling unit 21 supports the opening of the gantry 10 into which the subject P is inserted to be rotatable in a tilt angle direction.

The table 30 is an apparatus for placing and moving the subject P as a photographing target, and includes a base 31, a table driving circuitry 32, the table-top 33, and a support frame 34. The base 31 is a housing that supports the support frame 34 to be movable in a vertical direction. The table driving circuitry 32 is a driving mechanism that moves the table-top 33 on which the subject P is placed in a major axis direction of the table-top 33, and includes a motor, an actuator, and the like. The table-top 33 disposed on an upper surface of the support frame 34 is a plate on which the subject P is placed. The table driving circuitry 32 may move, in addition to the table-top 33, the support frame 34 in the major axis direction of the table-top 33.

The console 40 includes a memory 41, a display 42, the input interface circuitry 43, and processing circuitry 44. The console 40 will be described as an apparatus separated from the gantry 10, but the gantry 10 may include the console 40 or part of each constituent element of the console 40. For example, the memory 41 is implemented by a random access memory (RAM), a semiconductor memory element such as a flash memory, a hard disk, an optical disc, and the like. The memory 41 stores therein, for example, projection data and CT image data. For example, the memory 41 also stores therein a computer program for circuitry included in the X-ray CT apparatus 1 to implement various functions. The memory 41 may be implemented by a server group (cloud) connected to the X-ray CT apparatus 1 via a network.

The display 42 displays various pieces of information. For example, the display 42 displays various images generated by the processing circuitry 44, or displays a graphical user interface (GUI) for receiving various operations from an operator. For example, the display 42 is a liquid crystal display or a cathode ray tube (CRT) display. The display 42 may be a desktop type, or may be constituted of a tablet terminal and the like that can wirelessly communicate with a main body of the console 40. The display 42 is an example of a display unit.

The input interface circuitry 43 receives various input operations from the operator, and converts the received input operations into electric signals to be output to the processing circuitry 44. For example, the input interface circuitry 43 receives, from the operator, input operations of a scan condition, a reconstruction condition for reconstructing CT image data, an image processing condition for generating a postprocessed image from the CT image data, and the like.

The input interface circuitry 43 is implemented by a mouse, a keyboard, a trackball, a switch, a button, a joystick, a touch pad for performing input operation by touching an operation surface, a touch screen obtained by integrating a display screen and a touch pad, non-contact input circuitry using an optical sensor, voice input circuitry, or the like. The input interface circuitry 43 may be disposed on the gantry 10. The input interface circuitry 43 may also be constituted of a tablet terminal and the like that can wirelessly communicate with the main body of the console 40. The input interface circuitry 43 does not necessarily include a physical operation component such as a mouse and a keyboard. For example, examples of the input interface circuitry 43 include electric signal processing circuitry that receives an electric signal corresponding to the input operation from external input equipment that is disposed separately from the console 40, and outputs the electric signal to the processing circuitry 44.

The processing circuitry 44 controls an operation of the entire X-ray CT apparatus 1. The processing circuitry 44 includes, for example, a control function 441, a preprocessing function 442, a reconstruction processing function 443, an image processing function 444, and an injection control function 445. In the embodiment, respective processing functions executed by the control function 441, the preprocessing function 442, the reconstruction processing function 443, the image processing function 444, and the injection control function 445 are stored in the memory 41 in a form of a computer-executable computer program. The processing circuitry 44 is a processor that implements a function corresponding to each computer program by reading out from the memory 41 and executing the computer program. In other words, the processing circuitry 44 after reading out each computer program has each function illustrated in the processing circuitry 44 in FIG. 1.

In FIG. 1, the control function 441, the preprocessing function 442, the reconstruction processing function 443, the image processing function 444, and the injection control function 445 are assumed to be implemented by a single processor, but the processing circuitry 44 may be configured by combining a plurality of independent processors, and the functions may be implemented by executing computer programs by the respective processors. In FIG. 1, the single storage circuitry such as the memory 41 is assumed to store a computer program corresponding to each processing function, but a plurality of pieces of storage circuitry may be disposed in a dispersed manner, and the processing circuitry 44 may be configured to read out a corresponding computer program from an individual one of the pieces of the storage circuitry.

A word of “processor” used in the above description means, for example, a central processing unit (CPU), a graphical processing unit (GPU), or circuitry such as an application specific integrated circuit (ASIC) and a programmable logic device (for example, a simple programmable logic device (SPLD), a complex programmable logic device (CPLD), and a field programmable gate array (FPGA)). The processor implements a function by reading out and executing the computer program stored in the memory 41. Instead of storing a computer program in the memory 41, the computer program may be directly incorporated in circuitry of the processor. In this case, the processor implements a function by reading out and executing the computer program incorporated in the circuitry.

The control function 441 controls various kinds of processing based on an input operation received from the operator via the input interface circuitry 43. Specifically, the control function 441 controls a CT scan performed by the gantry 10. For example, the control function 441 controls processing of collecting counting results by the gantry 10 by controlling operations of the X-ray high-voltage apparatus 14, the X-ray detector 12, the control apparatus 15, the DAS 18, and the table driving circuitry 32. The control function 441 also performs control to cause various pieces of image data stored in the memory 41 to be displayed on the display 42. The control function 441 is an example of a control unit. The control function 441 also controls an operation of the stand 20 to rotate the gantry 10 in each tilt direction. The control function 441 also controls the operation of the stand 20 to move the gantry 10 in the vertical direction.

The preprocessing function 442 generates projection data by performing preprocessing such as logarithm conversion processing, offset correction processing, sensitivity correction processing between channels, and beam hardening correction on the detection data output from the DAS 18. The preprocessing function 442 is an example of a preprocessing unit.

The reconstruction processing function 443 generates CT image data by performing reconstruction processing on the projection data generated by the preprocessing function 442 using a filtered back projection method, a successive approximation reconstruction method, or the like. The reconstruction processing function 443 stores the reconstructed CT image data in the memory 41. The reconstruction processing function 443 is an example of a reconstruction processing unit.

The image processing function 444 converts the CT image data generated by the reconstruction processing function 443 into image data such as a tomogram of an optional cross section or a three-dimensional image obtained by rendering processing using a well-known method based on the input operation received from the operator via the input interface circuitry 43. The image processing function 444 stores the converted image data in the memory 41. The image processing function 444 is an example of an image processing unit.

The injection control function 445 controls injection of a medical fluid into the subject P by an injection apparatus 70 (refer to FIG. 8). For example, the medical fluid is a liquid such as a contrast medium or a physiological saline solution.

The following describes injection of the medical fluid into the subject P.

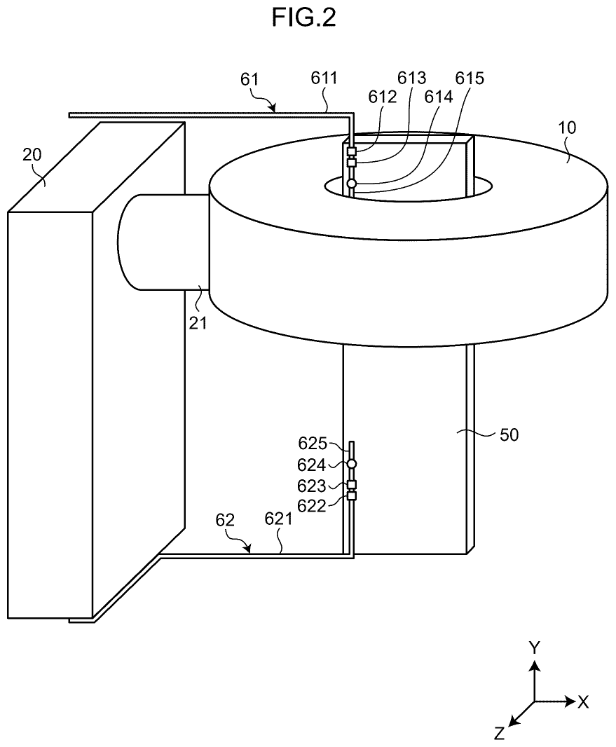

FIG. 2 is a diagram illustrating an example of an injection route of the medical fluid for the subject P in the X-ray CT apparatus 1 according to the first embodiment. FIG. 2 illustrates the injection route for the subject P in a standing position.

In some cases, it is difficult for the subject P to keep the standing position due to a disease, an injury, or the like. Thus, the X-ray CT apparatus 1 includes a subject supporting unit 50 that supports the subject P in the standing position. For example, the subject supporting unit 50 is a plate-shaped member erected to be substantially perpendicular to the floor surface. The subject supporting unit 50 is not limited to the plate-shaped member, but may be a bar-shaped member, a combination of the plate-shaped member and the bar-shaped member, or a member having another shape. Furthermore, in a case in which the subject P is in a sitting position, the subject supporting unit 50 may be a chair.

In a case of photographing the subject P, the stand 20 rotates the gantry 10 in a tilt direction by rotating the coupling unit 21. More specifically, the stand 20 rotates the coupling unit 21 so that the X-ray tube 11 and the X-ray detector 12 rotate around the subject supporting unit 50 that is substantially perpendicular to the floor surface. That is, the stand 20 rotates the coupling unit 21 so that the hollow of the gantry 10 into which the subject P is inserted is opposed to the floor surface.

The stand 20 moves the gantry 10 in the vertical direction in a state in which the hollow of the gantry 10 is opposed to the floor surface. While moving in the vertical direction, the gantry 10 photographs the subject P by helical scanning. The gantry 10 may photograph the subject P by non-helical scanning of alternately performing a scan of the subject P and movement of the table-top 33 instead of helical scanning, or may photograph the subject P by another method.

At the time of photographing the subject P, the X-ray CT apparatus 1 may inject a contrast medium into the subject P. The X-ray CT apparatus 1 includes an upper route 61 and a lower route 62 as injection paths for injecting the contrast medium into the subject P. That is, the X-ray CT apparatus 1 has an injection path for injecting a medical fluid into the subject P from at least one of an upper side and a lower side in the vertical direction of the gantry 10.

The upper route 61 is an injection path from the upper side of the X-ray CT apparatus 1 toward the subject P. The upper route 61 includes an apparatus-side tube 611, an air sensor 612, a connection sensor 613, a connection unit 614, and a subject-side tube 615.

The lower route 62 is an injection path from the lower side of the X-ray CT apparatus 1 toward the subject P. The lower route 62 includes an apparatus-side tube 621, an air sensor 622, a connection sensor 623, a connection unit 624, and a subject-side tube 625.

The apparatus-side tubes 611 and 621 are tubes connected to the injection apparatus 70 that injects the contrast medium and the like into the subject P. The medical fluid sent out from the injection apparatus 70 passes through the apparatus-side tubes 611 and 621.

For example, on the upper route 61, the apparatus-side tube 611 passes through a member disposed on the upper side of the X-ray CT apparatus 1 toward the subject P. On the lower route 62, the apparatus-side tube 621 passes through a member disposed on the lower side of the X-ray CT apparatus 1 toward the subject P.

The air sensor 612 detects air from at least one of the inside of the apparatus-side tube 611 and the inside of the subject-side tube 615. Similarly, the air sensor 622 detects air from at least one of the inside of the apparatus-side tube 621 and the inside of the subject-side tube 625. That is, the air sensors 612 and 622 detect air from the inside of at least one of the apparatus-side tubes 611 and 621 for injecting the medical fluid to the subject P on the injection paths, and the subject-side tubes 615 and 625. The air sensors 612 and 622 are examples of an air detection unit. For example, the air sensors 612 and 622 detect air by an optical sensor. The air sensors 612 and 622 are disposed on the subject supporting unit 50.

The connection sensor 613 detects that the subject-side tube 615 is connected to the connection unit 614. Similarly, the connection sensor 623 detects that the subject-side tube 625 is connected to the connection unit 624. The connection sensors 613 and 623 detect that the apparatus-side tubes 611 and 621 on a side of the injection apparatus 70 that injects the medical fluid into the subject P are connected to the subject-side tubes 615 and 625 on the subject side on the injection paths. The connection sensors 613 and 623 are examples of a connection detection unit. The apparatus-side tubes 611 and 621 are examples of a first tube. The subject-side tubes 615 and 625 are examples of a second tube.

For example, the connection sensors 613 and 623 detect connection of the subject-side tubes 615 and 625 by a microswitch. More specifically, the microswitch is disposed to be depressed by the subject-side tubes 615 and 625 when the subject-side tubes 615 and 625 are connected thereto. The connection sensors 613 and 623 detect connection of the subject-side tubes 615 and 625 depending on whether the microswitch is depressed by the subject-side tubes 615 and 625. The connection sensors 613 and 623 are disposed on the subject supporting unit 50.

The connection unit 614 is a member that connects the apparatus-side tube 611 with the subject-side tube 615. The connection unit 614 may be formed as part of the apparatus-side tube 611 or the subject-side tube 615, or may be an independent member.

Similarly, the connection unit 624 is a member that connects the apparatus-side tube 621 with the subject-side tube 625. The connection unit 624 may be formed as part of the apparatus-side tube 621 or the subject-side tube 625, or may be an independent member. The connection units 614 and 624 are disposed on the subject supporting unit 50.

The subject-side tube 615 is a tube that is connected to the subject P. That is, the subject-side tube 615 is a tube including a needle to be inserted into the subject P attached to an end on the subject P side thereof.

Similarly, the subject-side tube 625 is a tube that is connected to the subject P. That is, the subject-side tube 625 is a tube including a needle to be inserted into the subject P attached to an end on the subject P side thereof.

With such a configuration, the injection control function 445 controls injection of the contrast medium and the like by the injection apparatus 70.

More specifically, the injection control function 445 controls which of the upper route 61 as the injection path on the upper side in the vertical direction of the gantry 10 and the lower route 62 as the injection path on the lower side in the vertical direction of the gantry 10 is used for injecting the medical fluid into the subject P. The injection control function 445 is an example of an injection control unit. The upper route 61 is an example of a first injection path. The lower route 62 is an example of a second injection path. For example, the injection control function 445 controls which of the upper route 61 as the injection path from the upper side in the vertical direction of the gantry 10 toward the subject P and the lower route 62 as the injection path from the lower side in the vertical direction of the gantry 10 toward the subject P is used for injecting the medical fluid into the subject P based on a photographing condition for the subject P.

For example, the injection control function 445 controls which of the upper route 61 and the lower route 62 is used for injecting the medical fluid into the subject P based on a posture of the subject P at the time of photographing. For example, the injection control function 445 determines to inject the medical fluid into the subject P through the upper route 61 in a case of photographing the subject P in a posture of raising his/her hand. On the other hand, the injection control function 445 determines to inject the medical fluid into the subject P through the lower route 62 in a case of photographing the subject P in a posture of lowering his/her hand.

The injection control function 445 also notifies which of the upper route 61 and the lower route 62 is used for injecting the medical fluid into the subject P based on the photographing condition for the subject P by the X-ray tube 11 and the X-ray detector 12. The injection control function 445 is an example of an injection notification unit. That is, for example, the injection control function 445 displays that the medical fluid is injected into the subject P through the upper route 61 on the display 42. Alternatively, the injection control function 445 displays that the medical fluid is injected into the subject P through the lower route 62 on the display 42.

The injection control function 445 also controls injection of the medical fluid into the subject P based on respective detection results of the sensor disposed on the upper route 61 and the sensor disposed on the lower route 62.

More specifically, the injection control function 445 determines whether the injection path is available based on a detection result of the connection sensor 613. That is, the injection control function 445 determines whether the apparatus-side tube 611 is connected to the subject-side tube 615 to enable the medical fluid to flow to the subject P. The injection control function 445 does not inject the medical fluid into the subject P when the air sensor 612 detects air.

In a case of injecting the medical fluid into the subject P through the upper route 61, when the air sensor 612 does not detect air from the inside of the apparatus-side tube 611 or the inside of the subject-side tube 615, and the connection sensor 613 detects connection between the connection unit 614 and the subject-side tube 615, the injection control function 445 permits injection of the medical fluid through the upper route 61. In other words, when the air sensor 612 detects air from the inside of the apparatus-side tube 611 or the inside of the subject-side tube 615, or when the connection sensor 613 does not detect connection between the connection unit 614 and the subject-side tube 615, the injection control function 445 prohibits injection of the medical fluid through the upper route 61. For example, the injection control function 445 does not inject the medical fluid even if it receives an operation of instructing to start injection of the medical fluid.

When the air sensor 612 detects air from the inside of the apparatus-side tube 611 or the inside of the subject-side tube 615, the injection control function 445 may display the fact that air is detected on the display 42. When the connection sensor 613 does not detect connection between the connection unit 614 and the subject-side tube 615, the injection control function 445 may display the fact that the connection unit 614 is not connected to the subject-side tube 615 on the display 42.

The injection control function 445 also performs, for the lower route 62, control similar to that for the upper route 61.

More specifically, the injection control function 445 determines whether the injection path is available based on a detection result of the connection sensor 623. That is, the injection control function 445 determines whether the apparatus-side tube 621 is connected to the subject-side tube 625 to enable the medical fluid to flow to the subject P. The injection control function 445 does not inject the medical fluid into the subject P when the air sensor 622 detects air.

In a case of injecting the medical fluid into the subject P through the lower route 62, when the air sensor 622 does not detect air from the inside of the apparatus-side tube 621 or the inside of the subject-side tube 625, and the connection sensor 623 detects connection between the connection unit 624 and the subject-side tube 625, the injection control function 445 permits injection of the medical fluid through the lower route 62. In other words, when the air sensor 622 detects air from the inside of the apparatus-side tube 621 or the inside of the subject-side tube 625, or when the connection sensor 623 does not detect connection between the connection unit 624 and the subject-side tube 625, the injection control function 445 prohibits injection of the medical fluid through the lower route 62. For example, the injection control function 445 does not inject the medical fluid even if it receives an operation of instructing to start injection of the medical fluid.

When the air sensor 622 detects air from the inside of the apparatus-side tube 621 or the inside of the subject-side tube 625, the injection control function 445 may display the fact that air is detected on the display 42. When the connection sensor 623 does not detect connection between the connection unit 624 and the subject-side tube 625, the injection control function 445 may display the fact that the connection unit 624 is not connected to the subject-side tube 625 on the display 42.

As described above, the X-ray CT apparatus 1 according to the first embodiment includes the stand 20 that supports the gantry 10, and moves the gantry 10 in the vertical direction. The X-ray CT apparatus 1 includes the upper route 61 or the lower route 62 for injecting the medical fluid into the subject P from at least one of the upper side and the lower side in the vertical direction of the gantry 10.

For example, by using the upper route 61 in a case of a posture of raising a hand into which a needle for injecting the contrast medium is inserted, and using the lower route 62 in a case of a posture of lowering a hand into which a needle for injecting the contrast medium is inserted, the health professional can dispose the tube through which the contrast medium passes outside the photographing region. Thus, the X-ray CT apparatus 1 can provide a route through which the contrast medium can be injected into the subject P in a standing position or a sitting position.

### First Modification

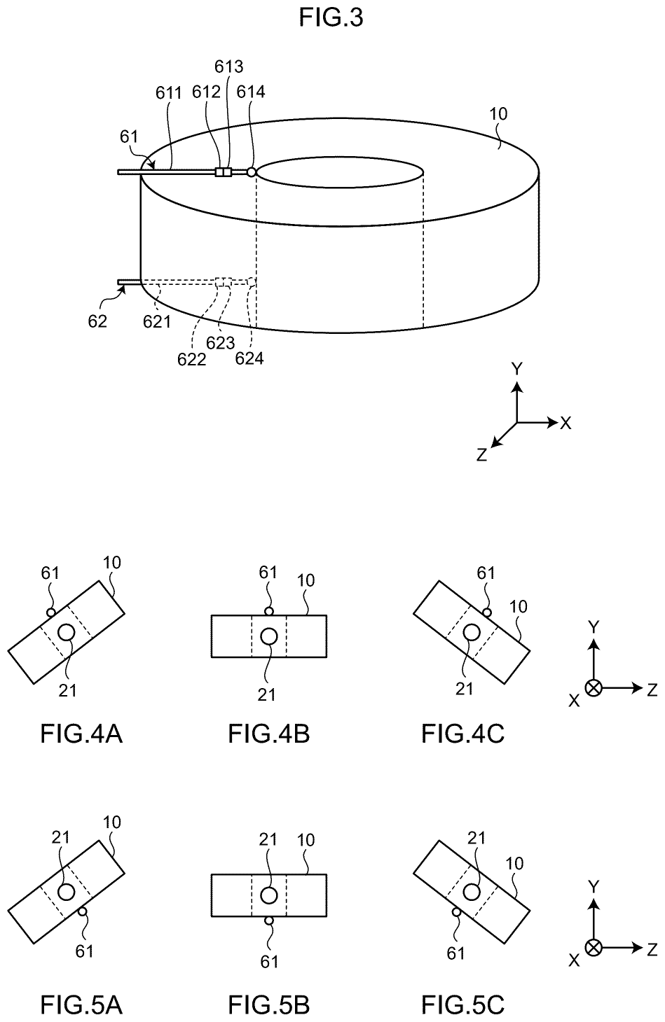

FIG. 3 is a diagram illustrating an example of the gantry 10 according to a first modification of the first embodiment. As illustrated in FIG. 3, the connection units 614 and 624 may be disposed on the gantry 10. The air sensors 612 and 622, and the connection sensors 613 and 623 may be disposed on the gantry 10.

More specifically, in a case in which an insertion direction of the subject P to be inserted into the gantry 10 is substantially parallel with the vertical direction, the air sensor 612, the connection sensor 613, and the connection unit 614 of the upper route 61 are disposed on the upper side of the gantry 10. On the other hand, the air sensor 622, the connection sensor 623, and the connection unit 624 of the lower route 62 are disposed on the lower side of the gantry 10.

Also in this case, the X-ray CT apparatus 1 injects the contrast medium into the subject P from the upper side or the lower side. Thus, the X-ray CT apparatus 1 can provide a route through which the contrast medium can be injected into the subject P in a standing position or a sitting position.

### Second Modification

The X-ray CT apparatus 1 according to a second modification of the first embodiment may include at least one of the upper route 61 and the lower route 62. That is, the X-ray CT apparatus 1 does not necessarily include the lower route 62.

FIGS. 4A to 4C are diagrams illustrating an example of the gantry 10 according to a second modification of the first embodiment. FIG. 4A illustrates a state in which the gantry 10 in FIG. 4B is rotated in a tilt direction. FIG. 4B illustrates a state in which the insertion direction of the subject P to be inserted into the gantry 10 is substantially parallel with the vertical direction, and the upper route 61 is disposed at a position opposed to a ceiling. FIG. 4C illustrates a state in which the gantry 10 in FIG. 4B is rotated in the tilt direction. Each of dispositions in FIGS. 4A, 4B, and 4C is a disposition in a case of photographing the subject P in a standing position or a sitting position. FIGS. 5A to 5C are diagrams illustrating an example of the gantry 10 according to the second modification of the first embodiment. FIG. 5A illustrates a state in which the gantry 10 in FIG. 5B is rotated in the tilt direction. FIG. 5B illustrates a state in which the insertion direction of the subject P to be inserted into the gantry 10 is substantially parallel with the vertical direction, and the upper route 61 is disposed at a position opposed to the floor surface. FIG. 5C illustrates a state in which the gantry 10 in FIG. 5B is rotated in the tilt direction. Each of dispositions in FIGS. 5A, 5B, and 5C is a disposition in a case of photographing the subject P in a standing position or a sitting position.

As illustrated in FIGS. 5A to 5C, the X-ray CT apparatus 1 rotates the coupling unit 21 to rotate the gantry 10. Due to this, the disposition of the upper route 61 becomes substantially the same as that of the lower route 62. Thus, the X-ray CT apparatus 1 can direct the subject-side tube 615 toward the subject P from the lower side of the X-ray CT apparatus 1 even when the lower route 62 is not included therein.

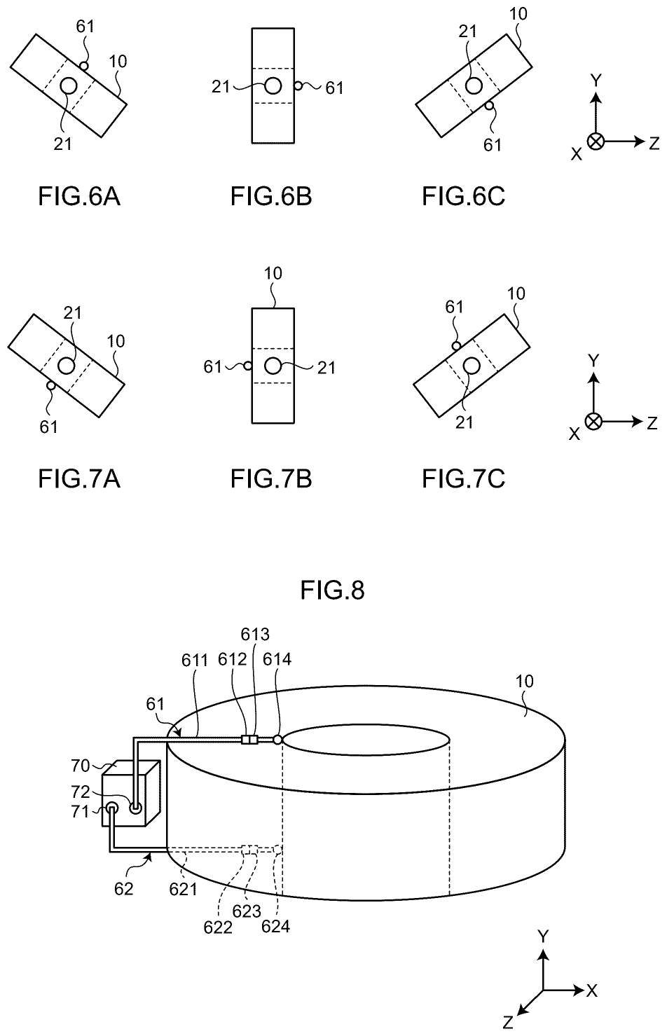

FIGS. 6A to 6C are diagrams illustrating an example of the gantry 10 according to the second modification of the first embodiment. FIG. 6A illustrates a state in which the gantry 10 in FIG. 6B is rotated in the tilt direction. FIG. 6B illustrates a state in which the insertion direction of the subject P to be inserted into the gantry 10 is substantially parallel with the floor surface, and the insertion direction of the subject P in the hollow of the gantry 10 is substantially parallel with the floor surface. FIG. 6C illustrates a state in which the gantry 10 in FIG. 6B is rotated in the tilt direction. Each of dispositions in FIGS. 6A, 6B, and 6C is a disposition in a case of photographing the subject P in a lying position.

FIGS. 7A to 7C are diagrams illustrating an example of the gantry 10 according to the second modification of the first embodiment. FIG. 7A illustrates a state in which the gantry 10 in FIG. 7B is rotated in the tilt direction. FIG. 7B illustrates a state in which the insertion direction of the subject P to be inserted into the gantry 10 is substantially parallel with the floor surface, and the insertion direction of the subject P in the hollow of the gantry 10 is substantially parallel with the floor surface, the gantry 10 being rotated in the opposite direction of FIG. 6B. FIG. 7C illustrates a state in which the gantry 10 in FIG. 7B is rotated in the tilt direction. Each of dispositions in FIGS. 7A, 7B, and 7C is a disposition in a case of photographing the subject P in a lying position.

As illustrated in FIGS. 7A to 7C, the X-ray CT apparatus 1 rotates the coupling unit 21 to rotate the gantry 10. Due to this, the X-ray CT apparatus 1 can inject the contrast medium into the subject P in a lying position to be photographed.

### Third Modification

The X-ray CT apparatus 1 according to a third modification of the first embodiment includes the lower route 62 of the upper route 61 and the lower route 62. That is, the X-ray CT apparatus 1 does not necessarily include the upper route 61.

### Second Embodiment

FIG. 8 is a diagram illustrating an example of a configuration of the injection apparatus 70 according to a second embodiment.

The injection apparatus 70 includes a container that houses one or a plurality of medical fluids. The injection apparatus 70 may also include one or a plurality of containers. The injection apparatus 70 includes a first output unit 71 and a second output unit 72. The first output unit 71 is connected to the apparatus-side tube 611 of the upper route 61. The second output unit 72 is connected to the apparatus-side tube 621 of the lower route 62. Due to this, the injection apparatus 70 includes the upper route 61 including the apparatus-side tube 611 connected to the container and the subject-side tube 615 for sending the medical fluid to the subject P. The injection apparatus 70 also includes the lower route 62 including the apparatus-side tube 621 connected to the container and the subject-side tube 625 for sending the medical fluid to the subject P.

For example, the injection apparatus 70 is supported by the gantry 10. The injection apparatus 70 is not necessarily supported by the gantry 10 but may be supported by the stand 20, or may be supported by another member (not illustrated). Furthermore, the injection apparatus 70 may be an apparatus separated from the X-ray CT apparatus 1.

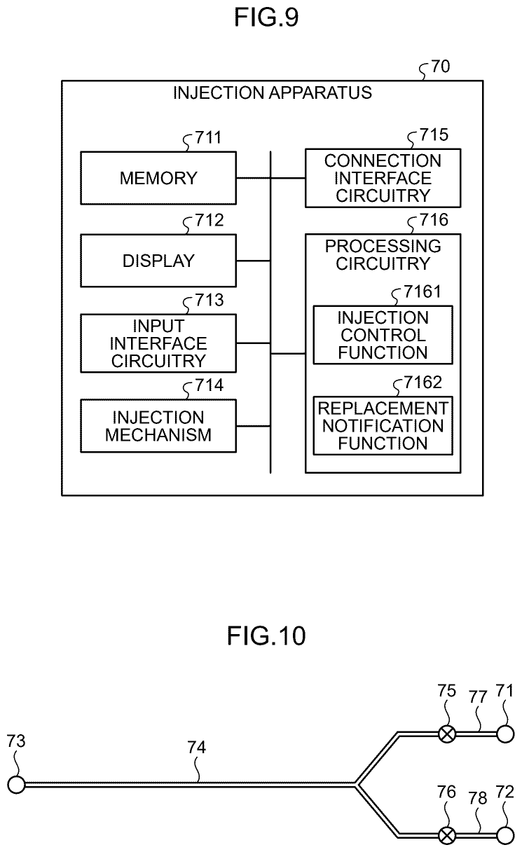

FIG. 9 is a diagram illustrating an example of the configuration of the injection apparatus 70. The injection apparatus 70 includes a memory 711, a display 712, an input interface circuitry 713, an injection mechanism 714, a connection interface circuitry 715, and processing circuitry 716.

For example, the memory 711 is implemented by a RAM, a semiconductor memory element such as a flash memory, a hard disk, an optical disc, and the like. The memory 711 stores therein, for example, projection data and CT image data. For example, the memory 711 also stores therein a computer program for circuitry included in the injection apparatus 70 to implement various functions. The memory 711 may be implemented by a server group (cloud) connected to the injection apparatus 70 via a network.

The display 712 displays various pieces of information. For example, the display 712 displays various images generated by the processing circuitry 716, or displays a GUI for receiving various operations from the operator. For example, the display 712 is a liquid crystal display or a CRT display. The display 712 may be a desktop type, or may be constituted of a tablet terminal and the like that can wirelessly communicate with a main body of the injection apparatus 70.

The input interface circuitry 713 is implemented by a mouse, a keyboard, a trackball, a switch, a button, a joystick, a touch pad for performing input operation by touching an operation surface, a touch screen obtained by integrating a display screen and a touch pad, non-contact input circuitry using an optical sensor, voice input circuitry, or the like. The input interface circuitry 713 may be disposed on the gantry 10. The input interface circuitry 713 may also be constituted of a tablet terminal and the like that can wirelessly communicate with the main body of the injection apparatus 70. The input interface circuitry 713 does not necessarily include a physical operation component such as a mouse and a keyboard. For example, examples of the input interface circuitry 713 include electric signal processing circuitry that receives an electric signal corresponding to an input operation from external input equipment that is disposed separately from the injection apparatus 70, and outputs the electric signal to the processing circuitry 716.

The injection mechanism 714 sends out a selected medical fluid from the container housing one or a plurality of medical fluids. For example, the injection mechanism 714 includes a pump and the like for sending out the medical fluid housed in the container.

The connection interface circuitry 715 controls communication and transmission of various kinds of data transmitted to or received from the X-ray CT apparatus 1. For example, the connection interface circuitry 715 is implemented by a network card, a network adapter, an NIC, and the like.

The processing circuitry 716 controls an operation of the entire injection apparatus 70. For example, the processing circuitry 716 executes an injection control function 7161 and a replacement notification function 7162. For example, respective processing functions executed by the injection control function 7161 and the replacement notification function 7162, which are constituent elements of the processing circuitry 716 illustrated in FIG. 9, are recorded in the memory 711 as computer-executable computer programs. The processing circuitry 716 is a processor, for example, and reads out, from the memory 711, and executes each computer program to implement a function corresponds to the read-out computer program. In other words, the processing circuitry 716 after reading out each computer program has each function illustrated in the processing circuitry 716 in FIG. 9.

The injection control function 7161 has the same function as that of the injection control function 445 according to the first embodiment. More specifically, the injection control function 7161 controls which of the upper route 61 and the lower route 62 is used for injecting the medical fluid into the subject P based on the photographing condition for the subject P acquired from the X-ray CT apparatus 1 via the connection interface circuitry 715. For example, when the subject P is in a standing position, the injection control function 7161 controls the injection apparatus 70 to send out the medical fluid from the first output unit 71 to the upper route 61. When the subject P is in a sitting position, the injection control function 7161 controls the injection apparatus 70 to send out the medical fluid from the second output unit 72 to the lower route 62.

The injection control function 7161 also controls injection of the medical fluid into the subject P based on respective detection results of the sensor disposed on the upper route 61 and the sensor disposed on the lower route 62.

The replacement notification function 7162 notifies about a replacement timing of the apparatus-side tubes 611 and 621. The replacement notification function 7162 is an example of a replacement notification unit. Herein, the subject-side tubes 615 and 625 are replaced for each subject P. On the other hand, the apparatus-side tubes 611 and 621 are not replaced for each subject P. Because they are not replaced for each subject P, the health professional needs to manage the replacement timing. Thus, replacement notification function 7162 notifies about the replacement timing of the apparatus-side tubes 611 and 621.

The replacement timing may be information indicating time, or may be information indicating the number of corresponding subjects P. For example, the replacement notification function 7162 notifies about the replacement timing of the apparatus-side tubes 611 and 621 when a set time has elapsed after the apparatus-side tubes 611 and 621 are connected. For example, the replacement notification function 7162 notifies about the replacement timing of the apparatus-side tubes 611 and 621 when the medical fluid is injected into a set number of the subjects P after the apparatus-side tubes 611 and 621 are connected. For example, the replacement notification function 7162 displays the replacement timing of the apparatus-side tubes 611 and 621 on the display 712. Moreover, when the test continues with the apparatus-side tubes 611 and 621 unreplaced, the replacement notification function 7162 may display a caution image to the operator that indicates replacement is required.

Next, the following describes a configuration of tubes from the container housing the medical fluid to the first output unit 71 or the second output unit 72 in the injection apparatus 70.

FIG. 10 is a diagram illustrating an example of a connection configuration of the tubes of the injection apparatus 70 according to the second embodiment; The injection apparatus 70 includes a mixing connection unit 73, an intermediate tube 74, a first valve 75, a first output tube 77, a second valve 76, and a second output tube 78.

The mixing connection unit 73 is connected to the container housing the medical fluid in which a physiological saline solution is mixed with a contrast medium. The intermediate tube 74 is a tube in which one end is connected to the mixing connection unit 73 and the other end is bifurcated. The other end of the intermediate tube 74 is connected to the first valve 75 and the second valve 76.

The first valve 75 opens or closes the tube between the intermediate tube 74 and the first output tube 77. That is, the first valve 75 switches whether to cause the medical fluid to flow to the first output unit 71. For example, the first valve 75 is opened when the medical fluid is discharged from the first output unit 71. The first output tube 77 is a tube that connects the first output unit 71 with the first valve 75.

The second valve 76 opens or closes the tube between the intermediate tube 74 and the second output tube 78. That is, the second valve 76 switches whether to cause the medical fluid to flow to the second output unit 72. For example, the second valve 76 is opened when the medical fluid is discharged from the second output unit 72. The second output tube 78 is a tube that connects the second output unit 72 with the second valve 76.

With this configuration, the injection apparatus 70 discharges the medical fluid from the first output unit 71 or the second output unit 72.

As described above, the injection apparatus 70 according to the second embodiment includes the first output unit 71 and the second output unit 72. The injection apparatus 70 also includes the upper route 61 from the upper side in the vertical direction of the gantry 10 toward the subject P, and the lower route 62 from the lower side in the vertical direction of the gantry 10 toward the subject P.

Due to this, by using the upper route 61 in a case of a posture of raising a hand into which a needle for injecting the contrast medium is inserted, and using the lower route 62 in a case of a posture of lowering a hand into which a needle for injecting the contrast medium is inserted, the health professional can dispose the tube through which the contrast medium passes outside the photographing region. Thus, the injection apparatus 70 can provide a route through which the contrast medium can be injected into the subject P in a standing position or a sitting position. The tube having the configuration illustrated in FIG. 10 may be disposed outside the injection apparatus 70. For example, the mixing connection unit 73 may be connected to an output unit of an injection apparatus including a single output unit.

### First Modification

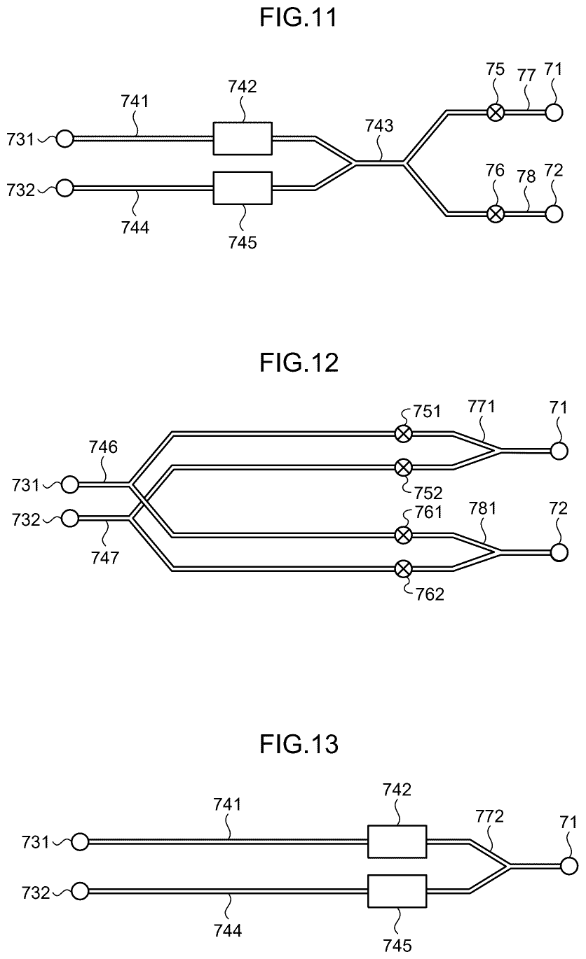

FIG. 11 is a diagram illustrating an example of a connection configuration of the tubes of the injection apparatus 70 according to a first modification of the second embodiment. The injection apparatus 70 may have the connection configuration of the tubes illustrated in FIG. 11. With the connection configuration of the tubes illustrated in FIG. 11, the injection apparatus 70 sends out the contrast medium and the physiological saline solution in a separated state to the vicinity of the first output unit 71 and the second output unit 72. Due to this, when a constitution ratio between the contrast medium and the physiological saline solution is changed, the injection apparatus 70 can output a mixed liquid having the changed constitution ratio more rapidly than the connection configuration of the tubes illustrated in FIG. 10. The tube having the configuration illustrated in FIG. 11 may be disposed outside the injection apparatus 70.

The injection apparatus 70 includes a contrast medium connection unit 731, a physiological saline solution connection unit 732, a first connection tube 741, a first check valve 742, a second connection tube 744, a second check valve 745, an intersecting tube 743, the first valve 75, the first output tube 77, the second valve 76, and the second output tube 78.

The contrast medium connection unit 731 is connected to a pump that sends out the contrast medium. The physiological saline solution connection unit 732 is connected to a pump that sends out the physiological saline solution.

The first connection tube 741 is a tube that connects the contrast medium connection unit 731 with the first check valve 742. The first check valve 742 is a valve that prevents backflow of the medical fluid. That is, the first check valve 742 is a valve that prevents the medical fluid in the intersecting tube 743 from flowing into the first connection tube 741.

The second connection tube 744 is a tube that connects the physiological saline solution connection unit 732 with the second check valve 745. The second check valve 745 is a valve that prevents backflow of the medical fluid. That is, the second check valve 745 is a valve that prevents the medical fluid in the intersecting tube 743 from flowing into the second connection tube 744.

The intersecting tube 743 is a tube into which the contrast medium flowing from the first check valve 742 and the physiological saline solution flowing from the second check valve 745 flow. The intersecting tube 743 is connected to the first valve 75 and the second valve 76.

The first valve 75 opens or closes the tube between the intersecting tube 743 and the first output tube 77. That is, the first valve 75 switches whether to cause the medical fluid to flow to the first output unit 71. For example, the first valve 75 is opened when the medical fluid is discharged from the first output unit 71. The first output tube 77 is a tube that connects the first output unit 71 with the first valve 75.

The second valve 76 opens or closes the tube between the intersecting tube 743 and the second output tube 78. That is, the second valve 76 switches whether to cause the medical fluid to flow to the second output unit 72. For example, the second valve 76 is opened when the medical fluid is discharged from the second output unit 72. The second output tube 78 is a tube that connects the second output unit 72 with the second valve 76.

### Second Modification

FIG. 12 is a diagram illustrating an example of a connection configuration of the tubes of the injection apparatus 70 according to a second modification of the second embodiment. The injection apparatus 70 may have the connection configuration of the tubes illustrated in FIG. 12. With the connection configuration of the tubes illustrated in FIG. 11, the injection apparatus 70 sends out the contrast medium and the physiological saline solution in a separated state to the vicinity of the first output unit 71 and the second output unit 72. Due to this, when a constitution ratio between the contrast medium and the physiological saline solution is changed, the injection apparatus 70 can output a mixed liquid having the changed constitution ratio more rapidly than the connection configuration of the tubes illustrated in FIG. 11. The tube having the configuration illustrated in FIG. 12 may be disposed outside the injection apparatus 70.

The injection apparatus 70 includes the contrast medium connection unit 731, the physiological saline solution connection unit 732, a first branched tube 746, a second branched tube 747, a third valve 751, a fourth valve 752, a fifth valve 761, a sixth valve 762, a first coupling tube 771, and a second coupling tube 781.

The contrast medium connection unit 731 is connected to a pump that sends out the contrast medium. The physiological saline solution connection unit 732 is connected to a pump that sends out the physiological saline solution.

The first branched tube 746 is a tube branched into two. One end of the first branched tube 746 is connected to the contrast medium connection unit 731. The other end of the first branched tube 746 is connected to the third valve 751 and the fifth valve 761.

The second branched tube 747 is a tube branched into two. One end of the second branched tube 747 is connected to the physiological saline solution connection unit 732. The other end of the second branched tube 747 is connected to the fourth valve 752 and the sixth valve 762.

The third valve 751 opens or closes the tube between the first branched tube 746 and the first coupling tube 771. That is, the third valve 751 switches whether to cause the medical fluid to flow to the first output unit 71. For example, the third valve 751 is opened when the medical fluid is discharged from the first output unit 71.

The fourth valve 752 opens or closes the tube between the second branched tube 747 and the first coupling tube 771. That is, the fourth valve 752 switches whether to cause the medical fluid to flow to the first output unit 71. For example, the fourth valve 752 is opened when the medical fluid is discharged from the first output unit 71.

The fifth valve 761 opens or closes the tube between the first branched tube 746 and the second coupling tube 781. That is, the fifth valve 761 switches whether to cause the medical fluid to flow to the second output unit 72. For example, the fifth valve 761 is opened when the medical fluid is discharged from the second output unit 72.

The sixth valve 762 opens or closes the tube between the second branched tube 747 and the third coupling tube 772. That is, the sixth valve 762 switches whether to cause the medical fluid to flow to the second output unit 72. For example, the sixth valve 762 is opened when the medical fluid is discharged from the second output unit 72.

The first coupling tube 771 is a tube into which the contrast medium flowing from the third valve 751 and the physiological saline solution flowing from the fourth valve 752 flow. The first coupling tube 771 is connected to the first output unit 71.

The second coupling tube 781 is a tube into which the contrast medium flowing from the fifth valve 761 and the physiological saline solution flowing from the sixth valve 762 flow. The second coupling tube 781 is connected to the second output unit 72.

### Third Modification

FIG. 13 is a diagram illustrating an example of a connection configuration of the tubes of the injection apparatus 70 according to a third modification of the second embodiment. The injection apparatus 70 may have the connection configuration of the tubes illustrated in FIG. 13. With the connection configuration of the tubes illustrated in FIG. 13, the injection apparatus 70 sends out the contrast medium and the physiological saline solution in a separated state to the vicinity of the first output unit 71 and the second output unit 72. Due to this, when a constitution ratio between the contrast medium and the physiological saline solution is changed, the injection apparatus 70 can output a mixed liquid having the changed constitution ratio more rapidly than the connection configuration of the tubes illustrated in FIG. 12. The tube having the configuration illustrated in FIG. 13 may be disposed outside the injection apparatus 70.

The injection apparatus 70 includes the contrast medium connection unit 731, the physiological saline solution connection unit 732, the first connection tube 741, the second connection tube 744, the first check valve 742, the second check valve 745, and the third coupling tube 772.

The contrast medium connection unit 731 is connected to a pump that sends out the contrast medium. The physiological saline solution connection unit 732 is connected to a pump that sends out the physiological saline solution.

The first connection tube 741 is a tube that connects the contrast medium connection unit 731 with the first check valve 742. The second connection tube 744 is a tube that connects the physiological saline solution connection unit 732 with the second check valve 745.

The first check valve 742 is a valve that prevents backflow of the medical fluid. That is, the first check valve 742 is a valve that prevents the medical fluid in the third coupling tube 772 from flowing into the first connection tube 741.

The second check valve 745 is a valve that prevents backflow of the medical fluid. That is, the second check valve 745 is a valve that prevents the medical fluid in the third coupling tube 772 from flowing into the second connection tube 744.

The third coupling tube 772 is a tube into which the contrast medium from the first check valve 742 and the physiological saline solution from the second check valve 745 flow. The third coupling tube 772 is connected to the first output unit 71.

### Fourth Modification

The health professional also replaces the apparatus-side tube 611 of the upper route 61. Herein, the apparatus-side tube 611 runs from the upper side of the X-ray CT apparatus 1 toward the subject P. That is, the apparatus-side tube 611 is disposed at a high position. Due to this, it is difficult for the health professional to replace the apparatus-side tube 611. Thus, a replacement assisting member 63 for assisting replacement of the apparatus-side tube 611 may be attached to the apparatus-side tube 611.

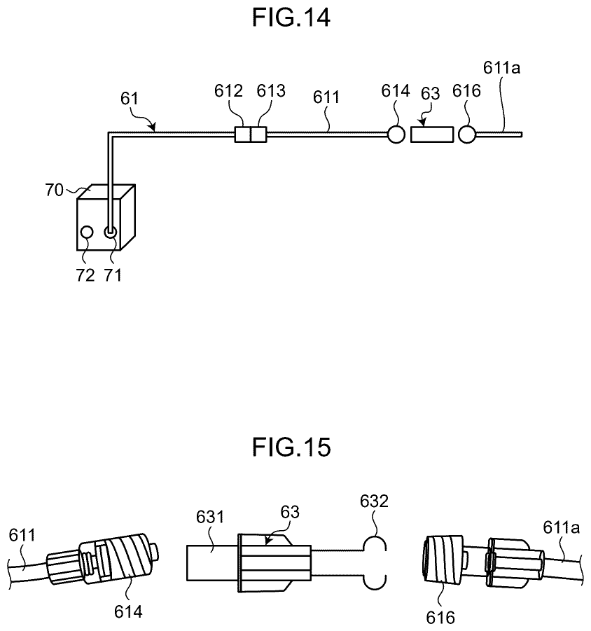

FIG. 14 is a diagram illustrating an example of attachment of the replacement assisting member 63 according to a fourth modification of the second embodiment. The replacement assisting member 63 is a member that assists replacement of the apparatus-side tube 611 after use with a new apparatus-side tube 611a. The replacement assisting member 63 is an example of a coupling member.

More specifically, the apparatus-side tube 611 after use is connected to the replacement assisting member 63. The replacement assisting member 63 is also connected to the new apparatus-side tube 611a. That is, the replacement assisting member 63 couples the apparatus-side tube 611 after use with the new apparatus-side tube 611a. The health professional pulls the apparatus-side tube 611 after use from the injection apparatus 70 side in a state in which the apparatus-side tube 611 after use is coupled with the new apparatus-side tube 611a.

A front end of the new apparatus-side tube 611a is connected to the apparatus-side tube 611 after use, so that the front end reaches the injection apparatus 70 through the path in which the apparatus-side tube 611 after use has been disposed. Accordingly, the health professional can replace the apparatus-side tube 611 after use with the new apparatus-side tube 611a. Due to this, the health professional can easily replace the apparatus-side tube 611 with the new apparatus-side tube 611a even when the apparatus-side tube 611 is disposed at a high position.

Next, the following describes details about the replacement assisting member 63. FIG. 15 is a diagram illustrating an example of external appearance of the replacement assisting member 63.

The apparatus-side tube 611 includes the connection unit 614 at an end on the subject P side. The connection unit 614 has an opening into which the subject-side tube 615 is inserted.

Herein, the apparatus-side tube 611 after use houses the medical fluid that has flowed in the apparatus-side tube 611. Thus, the replacement assisting member 63 includes a closing unit 631 that closes the end on the subject P side of the apparatus-side tube 611. The closing unit 631 is inserted into the opening of the connection unit 614 to close the connection unit 614.

The new apparatus-side tube 611a needs to maintain a clean state. Thus, the replacement assisting member 63 includes a holding unit 632 that holds an outer side of the new apparatus-side tube 611a. The holding unit 632 holds the outer side of the new apparatus-side tube 611a, so that a state of an inner side can be maintained.

According to at least one of the embodiments and the like described above, it is possible to provide a route through which the contrast medium can be injected into the subject P in a standing position or a sitting position.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

1. An X-ray CT apparatus comprising:
a gantry including a hollow into which a subject is inserted, an X-ray tube configured to emit X-rays to the hollow, and an X-ray detector configured to detect X-rays passed through the hollow;
a supporter configured to support the gantry and move the gantry in a vertical direction; and
an injection path through which a medical fluid is injected into the subject from at least one of an upper side and a lower side in the vertical direction of the gantry.

2. The X-ray CT apparatus according to claim 1, wherein
the supporter includes a coupler configured to be coupled with the gantry, and
the coupler rotates the gantry in a tilt direction.

3. The X-ray CT apparatus according to claim 1, further comprising processing circuitry configured to control which of a first injection path as an injection path on an upper side in the vertical direction of the gantry and a second injection path as an injection path on a lower side in the vertical direction of the gantry is used for injecting a medical fluid into the subject.

4. The X-ray CT apparatus according to claim 3, wherein
the processing circuitry
detects that a first tube on a side of an injection apparatus configured to inject the medical fluid into the subject is connected to a second tube on the subject side on the injection path, and
determines whether the injection path is available based on detection results of the first tube and the second tube.

5. The X-ray CT apparatus according to claim 3, wherein
the processing circuitry
detects air from an inside of at least one of a first tube on a side of an injection apparatus configured to inject the medical fluid into the subject and a second tube on the subject side on the injection path, and
does not inject the medical fluid into the subject when air is detected from the inside of at least one of the first tube and the second tube.

6. The X-ray CT apparatus according to claim 1, further comprising processing circuitry configured to notify which of a first injection path as an injection path on an upper side in the vertical direction of the gantry and a second injection path as an injection path on a lower side in the vertical direction of the gantry is used for injecting the medical fluid into the subject based on a photographing condition for the subject by the X-ray tube and the X-ray detector.

7. The X-ray CT apparatus according to claim 1, wherein the supporter supports an injection apparatus configured to inject the medical fluid into the subject.

8. An injection apparatus comprising:
a container configured to house one or a plurality of medical fluids;
a first injection path including a first tube connected to the container and a second tube configured to send the medical fluid to a subject; and
a second injection path including a first tube connected to the container and a second tube configured to send the medical fluid to the subject.

9. The injection apparatus according to claim 8, further comprising processing circuitry configured to notify about a replacement timing of the first tube.

10. The injection apparatus according to claim 8, wherein
the first tube is connected to a coupling member, and
the coupling member closes an end of the first tube after use on a side connected to the second tube, and holds an outer side of an end of the first tube to be used on a side connected to the container.
